(19) United States
(12) Patent Application Publication
(10) Pub. No.: US 2025/0312409 A1
(43) Pub. Date: Oct. 9, 2025

(54) METHODS OF TREATING DILATED CARDIOMYOPATHY AND HEART FAILURE

(71) Applicant: ARIZONA BOARD OF REGENTS ON BEHALF OF THE UNIVERSITY OF ARIZONA, Tucson, AZ (US)

(72) Inventors: Guy Reed, Phoenix, AZ (US); Inna Gladysheva, Phoenix, AZ (US); Ryan Sullivan, Phoenix, AZ (US)

(21) Appl. No.: 18/865,123

(22) PCT Filed: May 10, 2023
(86) PCT No.: PCT/US2023/021650

§ 371 Date: Nov. 12, 2024

Related U.S. Application Data
(60) Provisional application No. 63/340,133, filed on May 10, 2022.

(51) Int. Cl. A61K 38/09

(52) U.S. Cl. CPC A61K 38/09

(57) ABSTRACT

Methods for repurposing a therapeutically effective amount of drugs that reduce circulating testosterone levels and/or activity (androgen-deprivation drugs) for treating, ameliorating, and/or preventing progression of dilated cardiomyopathy and heart failure. Reduction in circulating testosterone levels and/or activity is effective for treating, ameliorating, and/or preventing progression of dilated cardiomyopathy and/or heart failure in the subject.

### CROSS-REFERENCES TO RELATED APPLICATIONS

This application claims benefit of U.S. Provisional Application No. 63/340,133 filed May 10, 2022, the specification of which is incorporated herein in its entirety by reference.

### FIELD OF THE INVENTION

The present invention relates to treating dilated cardiomyopathy and heart failure, in particular, to methods and compositions featuring testosterone lowering and/or testosterone suppression for the treatment and/or prevention of dilated cardiomyopathy progression and heart failure with reduced ejection fraction.

### BACKGROUND OF THE INVENTION

Approximately 6.2 million Americans have heart failure (HF) and the number is expected to double by 2035. Dilated cardiomyopathy (DCM), one of the major causes of HF, is characterized by progressive heart enlargement with a reduced ejection fraction (rEF). Despite the best available therapies, DCM progresses inexorably to symptomatic HFrEF with progressive fluid-salt retention (edema), breathlessness, fatigue, cachexia/sarcopenia, disability, and a mortality rate of about 50% within five years of diagnosis. New therapeutic strategies are needed to block the progression of DCM to symptomatic HF, improve the quality and prolong life.

Inventors surprisingly discovered that a reduction of plasma testosterone (T) levels in a pre-clinical DCM model beneficially altered transcription of heart contractile protein genes, improved systolic dysfunction, attenuated cardiac remodeling, edema and symptomatic HF, and prolonged life.

### BRIEF SUMMARY OF THE INVENTION

The present invention features methods and compositions that reduce circulating T levels by repurposing androgen-deprivation (T suppressing) drugs for treating, ameliorating, and/or preventing DCM progression and HF. The reduction in T may preserve systolic function, slow cardiac remodeling, fibrosis and edema, and development of symptomatic HF with reduced ejection fraction (HFrEF).

For example, the present invention provides methods for treating, ameliorating, or preventing progression of DCM and HF and/or development of DCM and/or development of HF in a subject in need thereof, wherein the method comprises administering to the subject a therapeutically effective amount of a drug that reduces circulating T levels, wherein reduction in circulating T levels is effective for treating, ameliorating, and/or preventing progression of DCM and HF in the subject.

The present invention also provides methods for treating, ameliorating, and/or preventing progression of DCM and development of HF in a subject in need thereof, wherein the method comprises administering to the subject a therapeutically effective amount of a gonadotropin releasing hormone (GnRH) antagonist, wherein the GnRH antagonist reduces circulating T levels and treats, ameliorates, or prevents progression of DCM and development of HF in the subject.

In some embodiments, the drug is degarelix, abarelix, cetrorelix, ganirelix, elagolix, or relugolix. In some embodiments, the drug is leuprorelin, goserelin, triptorelin, histrelin, or buserelin. The present invention is not limited to the aforementioned drugs or drug classes and includes other classes of androgen (T) supressants including androgen agonists and androgen receptor-antagonists and androgen synthesis inhibitors and combined androgen blockade by several classes of androgen (T) suppressants.

In some embodiments, the present invention may comprise a composition which may be formulated for oral or topical administration, or for intramuscular, subcutaneous, or intravenous injections. Without wishing to limit the invention to any theory or mechanism, the present invention may be effective for treating and/or preventing DCM and/or HF, or a combination thereof.

According to some embodiments, the present invention features a method of treating and/or preventing DCM and/or HF. The method may comprise administering to the subject a therapeutic amount of a T-inhibiting drug, and/or any of the compositions described herein.

In some embodiments, the drugs and/or composition described herein, including as used in the methods described herein, can be administered orally, topically, parenterally, intramuscularly, subcutaneously, or by intravenous injections.

One of the unique and inventive technical features of the present invention is the use of androgen-deprivation and/or T-lowering drugs to treat DCM and/or HF. Without wishing to limit the invention to any theory or mechanism, it is believed that the technical feature of the present invention advantageously provides for the treatment, amelioration, and/or prevention of progression of DCM and/or HF. None of the presently known prior references or work has the unique inventive technical feature of the present invention. Furthermore, the prior references teach away from the present invention. For example, as discussed below, previous research has indicated that T deficiency is believed to be deleterious in HF and T supplementation therapy is beneficial in HF. Furthermore, the inventive technical features of the present invention contributed to a surprising result. For example, because previous research has indicated that T deficiency is deleterious in HF and T supplementation therapy is beneficial in HF, T-lowering in HF was not anticipated to be beneficial. Surprisingly, the present invention provides benefits in DCM and/or HF via the use of androgen-deprivation and/or T-lowering drugs.

The effects of endogenous and exogenous T on cardiac pathophysiology are not fully understood and, as a result, are controversial. For example, Salzano et al. found that T deficiency plays a pivotal role in HF related to impaired cardiac performance and ultimately leads to a poor prognosis. Salzano et al. concluded that T replacement therapy is safe and effective and that hormone levels should be monitored in all HF patients. Andrea Salzano, Roberta D'Assante, Mark Lander, Michele Arcopinto, Eduardo Bossone, Toru Suzuki, Antonio Cittadini. Hormonal Replacement Therapy in Heart Failure: Focus on Growth Hormone and Testosterone. Heart Failure Clin 15 (2019) 377-391. https://doi.org/10.1016/j.bfc.2019.02.007

Conversely, inventors unexpectedly discovered that in the established progressive preclinical model of dilated cardiomyopathy (DCM), T-lowering therapy successfully attenuates progression of DCM and prevents development of symptomatic heart failure (HF). These findings are surprising as T deficiency is believed to be deleterious in HF and T supplementation therapy is beneficial in HF. See, for example, Salzano et. al. T-lowering in HF was not anticipated to be beneficial, but inventors surprisingly found a beneficial, not deleterious, effect.

In another study consistent with the existing cardiac-testosterone paradigm, Kirby et. al. found that T deficiency increases the risk of cardiovascular disease. T replacement therapy in Chronic Heart Failure (CHF) patients was shown to improve exercise capacity and reduce the risk of heart attack. Low T levels in longitudinal studies showed the highest cardiovascular and overall mortality. Critically, Kirby et. al. found that reductions in circulating T levels predict deterioration of functional capacity in CHF.

In contrast to the existing cardiac-testosterone paradigm, inventors' data suggests that lowering T levels (via medical reduction and the use of androgen-deprivation and/or T-lowering drugs) actually improves cardiac performance and HF outcomes. Inventors' model tested a single agent (T deprivation), without concomitant medications or comorbidities, which are common complications and confounding variables identified in previous studies. Inventors' findings support the idea that hormone levels in HF patients should be monitored, particularly in the case of patients being treated with hormone-modulating therapy, and more significantly support the idea that medically-induced reduction in circulating T levels and/or T activity is effective for treating, ameliorating, and/or preventing progression of DCM and/or HF.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows lowering testosterone (T) levels improves contractile function in male dilated cardiomyopathy (DCM) mice. Ejection fraction (EF, %) was analyzed in littermate DCM male mice (on C57BL/6J background) at 20 weeks of age corresponding to Stage D heart failure (HF) in non-castrated DCM mice. Groups: Normal T (100%)—non castrated DCM mice (left bar); Low T (˜17%)—castrated DCM mice (middle bar); Reduced T (˜60%)—castrated DCM mice with partially restored T (right bar). Dotted line: level in WT congenic group (n=8). T plasma levels analyzed by ELISA. Data analyzed by One-way-ANOVA with Tukey's multiple comparison test and represent mean+/−SEM; *=between DCM groups; **P<0.001; +=DCM groups vs. WT group: ++++P<0.0001.

FIGS. 2A-2B show lowering testosterone (T) levels increases cGMP levels in plasma (FIG. 2A) and cytosol fraction of cardiac LV extract (FIG. 2B) of male DCM mice. cGMP was analyzed in littermate mice by ELISA; data were normalized to 100 ug of total protein in extracts. Groups: at 20 weeks of age=Stage D heart failure (HF) in non-castrated DCM mice. Normal T (100%)—non castrated DCM mice (left bar); Low T (˜17%)—castrated DCM mice (middle bar); Reduced T (˜60%)—castrated DCM mice with partially restored T (right bar). Dotted line: level in WT congenic group (n=8). T plasma levels analyzed by ELISA. Data analyzed by One-way-ANOVA with Tukey's multiple comparison test and represent mean+/−SEM; *=between DCM groups; **P<0.001; +=DCM groups vs. WT group: ++++P<0.0001.

FIG. 3 shows that lowering testosterone (T) levels prolongs survival in male DCM mice. Shown are Kaplan-Meir survival curves for DCM mice. Groups shown are as follows: Normal T (100%)—non-castrated DCM mice (black); Low T (˜17%) DCM mice; Blue open circle—castrated at 4 weeks of age or Stage A; Blue closed circle—castrated at 7 weeks of age or Stage B; Survival of control mice without DCM were normal and consistent with wild-type C57BL6/J mice. Normal T vs. Low T: **P<0.001; Low T (closed) vs. Low T (open): **P<0.001.

FIGS. 4A-4D show lowering testosterone (T) level by T suppressing drug degarelix treatment improves contractile function (FIG. 4A), reduces pathologically elevated heart (FIG. 4B) and lung edema (FIGS. 4C, 4D), and attenuates HF in male DCM mice. Ejection fraction (EF, %) (FIG. 4A), heart-to-body weight ratios, HW/BW (FIG. 4B), lung-to-body weight ratios, LW/BW, (FIG. 4C) and pleural effusion, (FIG. 4D) were analyzed in male mice with DCM and WT control mice (C57BL/6J) at 20 weeks of age corresponding to Stage D HF in non-treated DCM mice. Groups: Normal non-T-treated DCM mice (black, left bar); Low T (˜20%) DCM mice treated with degarelix, 15 mg/kg subcutaneous injection every 4 weeks (blue, right bar). Dotted line: Levels in WT congenic group (n=8). T plasma level analyzed by ELISA. Data (FIG. 4A-4C) analyzed by One-way-ANOVA with Tukey's multiple comparison test and represent mean t SEM. (FIG. 4D) analyzed by Fisher exact test. Number of experimental mice demonstrated on the graphs. *=between DCM groups: *P<0.05; **P<0.01; ***P<0.001; DCM groups vs. WT group: ++++P<0.0001; +++P<0.001.

FIGS. 5A-5F show lowering testosterone (T) reduces plasma norepinephrine, plasma renin activity (PRA), and pro-renin receptor ((P)RR) levels in male DCM mice. Groups: at 20 weeks of age=Stage D HF in non-castrated DCM mice. Normal T (100%) non-treated DCM mice (black, left bar); Low T (˜17%) castrated or degarelix-treated DCM mice (blue, right bar); Dotted line: Levels in WT congenic group (n=6-8). T plasma level analyzed by ELISA. Data analyzed by One-way-ANOVA with Tukey's multiple comparison test and represented as mean f SEM; +++P<0.001 in DCM vs. WT group; ****P<0.0001 in castrated vs non-castrated DCM groups. FIG. 5A shows lowering norepinephrine plasma levels by pharmacological T-lowering with degarelix. FIG. 5B shows normalization of PRA levels in DCM mice by surgical testosterone lowering. FIG. 5C shows normalization of PRA levels in DCM mice by pharmacological (degarelix) T-lowering. FIG. 5D shows normalization of (P)RR levels in DCM mice by surgical testosterone-lowering. FIG. 5E shows normalization of (P)RR levels in DCM mice by pharmacological (degarelix) T-lowering. FIG. 5F shows the impact of pharmacological testosterone lowering with degarelix on total levels of renin in plasma. Plasma biomarkers levels were analyzed in littermate mice with and without DCM.

FIGS. 6A-B show lowering testosterone (T) level by degarelix treatment increases plasma levels of ANP (FIG. 6A) and cGMP (FIG. 6B) in male DCM mice. Plasma ANP levels (analyzed as NT-ANP) and cGMP levels were analyzed by Elisa in littermate male DCM mice and WT mice (C57BL/6J) at 20 weeks of age corresponding to Stage D HF in non-treated DCM mice. Groups: Normal T, non-treated DCM mice (black, left bar); Low T (˜20%), DCM mice treated with degarelix, 15 mg/kg subcutaneous injection every 4 weeks (blue, right bar). Dotted line: level in WT congenic group (n=6). T plasma level analyzed by ELISA. Data analyzed by One-way-ANOVA with Tukey's multiple comparison test and represent mean t SEM; *=between DCM groups: **P<0.01; DCM groups vs. WT group: ++++P<0.0001; +++P<0.001.

FIG. 7 shows the effect of partial testosterone (T) reduction by ovariectomy on contractile function in female DCM mice. Ejection fraction (EF, %) was analyzed in littermate female mice (C57BL/6J) at 13 weeks of age corresponding to Stage D HF in non-ovariectomized female DCM mice. Groups: Normal T (100%), non-ovariectomized DCM mice (black); Reduced T (˜55%), ovariectomized DCM mice (red). Dotted line: EF level in WT congenic group (n=8). T plasma level analyzed by ELISA. EF data analyzed by One-way-ANOVA with Tukey's multiple comparison test and represent mean t SEM; DCM vs. WT: ++++P<0.0001.

### DETAILED DESCRIPTION OF THE INVENTION

Before the present compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

As defined herein, the terms “treating” or “treatment” of a condition includes: (1) preventing the condition, i.e., causing the clinical symptoms of the condition not to develop in a mammal that may be exposed to or predisposed to the condition but does not yet experience or display symptoms of the condition; (2) inhibiting the condition, i.e., arresting or reducing the development of the condition or its clinical symptoms; or (3) ameliorating or relieving the condition, i.e., causing regression of the condition or its clinical symptoms. As used herein, the terms “treat” or “treatment” refer to both therapeutic treatment or preventative measures, wherein the object is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the patient may be identified (e.g., diagnosed) as one suffering from the disease or condition prior to administration of the composition of the invention.

A “therapeutically effective amount” refers to an amount that is sufficient to achieve the desired therapeutic result or to have an effect on undesired symptoms but is generally insufficient to cause intolerable adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific composition employed; the duration of the treatment; drugs used in combination or coincidental with the specific composition employed and like factors well known in the medical arts. For example, it is well within the skill of the art to start doses of the composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As used herein, “clinical improvement” may refer to a noticeable reduction in the symptoms of a disorder, or cessation thereof.

As used herein, the terms “administering” or “administer” is defined as the introduction of a substance (composition) into cells in vitro or into the body of an individual in vivo and includes topical, oral, nasal, ocular, rectal, vaginal and parenteral routes. The composition of the present invention may be administered via any route of administration including, but not limited to orally, sublingually, parenterally (e.g., intravenously and subcutaneously), by intramuscular injection, topically (including ophthalmically, vaginally, rectally, intranasally), by intraperitoneal injection, intrathecally, transdermally, extracorporeally, intradermally or the like. The disclosed compositions can be administered topically, orally, intravenously, intraperitoneally, intramuscularly, subcutaneously, intracavity, transdermally, sublingually or through buccal delivery.

A composition can also be administered by buccal delivery or by sublingual delivery. As used herein “buccal delivery” may refer to a method of administration in which the composition is delivered through the mucosal membranes lining the cheeks. In some embodiment, for a buccal delivery the composition is placed between the gum and the cheek of a patient. As used herein “sublingual delivery” may refer to a method of administration in which the composition is delivered through the mucosal membrane under the tongue. In some embodiments, for a sublingual delivery the composition is administered under the tongue of a patient.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, for example, U.S. Pat. No. 3,610,795, which is incorporated by reference herein.

As used herein, the terms “% wt” and “% w/v” can be used interchangeably and are defined as a % concentration of unit weight or mass to unit volume. For example, a % w/v may refer to a concentration in g/ml.

As used herein, the terms “% vol”, “% vol/vol” and “% v/v” can be used interchangeably and refer to a volume percentage of a component relative to the total volume of the solution or mixture. For example, 5% vol of component A may refer to 5 ml of component A to 100 ml of total volume of the mixture.

Any of the minerals disclosed herein may be used in the form of pharmaceutically acceptable salts. As used herein, “pharmaceutically acceptable” is meant that which is useful for the preparation of a pharmaceutical composition and is generally safe, non-toxic and neither biologically nor otherwise undesirable and which is acceptable for veterinary use as well as in human pharmaceutics.

By “pharmaceutically acceptable salts” of a composition is meant salts which are pharmaceutically acceptable as defined herein and which have the desired pharmacological action of the parent compound. Such salts comprise useful salts are acid addition salts, which are formed by pharmaceutically acceptable free acids. The acid addition salts are obtained from inorganic acid, such as hydrochloric acid, nitric acid, phosphoric acid, sulfuric acid, hydrobromic acid, hydroiodic acid, nitrous acid or phosphorous acid and the like; or formed with pharmaceutically acceptable organic acids, such as aliphatic mono- and dicarboxylates, phenyl-substituted alkanoates, hydroxyalkanoates, alkanedioates, aromatic acids, aliphatic and aromatic sulphonic acids, acetic acid, benzenesulfonic acid, benzoic acid, camphorsulfonic acid, citric acid, ethane-sulfonic acid, fumaric acid, glucoheptonic acid, gluconic acid, glutamic acid, glycolic acid, hydroxynaphthoic acid, 2-hydroxyethanesulfonic acid, lactic acid, maleic acid, malic acid, mandelic acid, methanesulfonic acid, muconic acid, 2-naphthalenesulfonic acid, propionic acid, salicylic acid, succinic acid, dibenzoyl-L-tartaric acid, tartaric acid, p-toluenesulfonic acid, trimethylacetic acid, trifluoroacetic acid and the like. Such pharmaceutically nontoxic salts include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitro benzoate, hydroxybenzoate, methoxybenzoate, phthalate, terephthalate, benzene sulfonate, toluene sulfonate, chlorobenzene sulfonate, xylenesulfonate, phenyl acetate, phenylpropionate, phenylbutyrate, citrate, lactate, hydroxybutyrate, glycolate, malate, tartrate, methane sulfonate, propane sulfonate, naphthalene-1-sulfonate, naphthalene-2-sulfonate or mandelate.

In other embodiments, the pharmaceutically acceptable salts may comprise the addition salts of pharmaceutically acceptable bases formed when an acid proton contained in the parent compound is either replaced by a metal ion e.g. an alkaline metal ion, an alkaline-earth metal ion or aluminum ion; or coordinated with a pharmaceutically acceptable organic or inorganic base. Acceptable organic bases include diethanolamine, ethanolamine, N-methylglucamine, triethanolamine, tromethamine and the like. Acceptable inorganic bases include aluminum hydroxide, calcium hydroxide, potassium hydroxide, sodium carbonate and sodium hydroxide.

In some embodiments, the compositions described herein may include bases that can encourage timed release. Non-limiting examples of such bases include hydrogels, micelles, vesicles, nanoparticles, hydroxypropyl methylcellulose (HPMC), poly(ethylene glycol) (PEG), polylactides (PLA), polyglycolides (PGA), poly(lactide-co-glycolides) (PLGA), polyanhydrides, and polyorthoesters.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

As used herein, the terms “those defined above” and “those defined herein” when referring to a variable incorporates by reference the broad definition of the variable as well as any narrow and/or preferred definitions, if any.

In some embodiments, the aforementioned compositions are administered via injection. In other embodiments, the aforementioned compositions are administered sublingually. In other embodiments, the aforementioned compositions are administered topically. In other embodiments, the aforementioned compositions are administered orally or intranasally.

In some embodiments, compositions described herein may be administered once daily or twice daily. In another embodiment, compositions described herein may be administered at least once to four times daily. In some embodiments, compositions described herein may be administered at least once daily, at least once every other day, at least once weekly, or once, twice, or 3 times per week. In some embodiments, compositions described herein may be administered once every two weeks, once every three weeks, once every four weeks, once every five weeks, once every six weeks, once every seven weeks, once every eight weeks, once every nine weeks, once every ten weeks, once every 11 weeks, once every 12 weeks, or the like.

In some embodiments, the compositions described herein are administered orally. In other embodiments, compositions described herein are administered sublingually. In further embodiments, compositions described herein are administered parenterally (e.g., intravenously and subcutaneously). In some embodiments, compositions described herein are administered by intramuscular injection (i.e., intramuscularly). In other embodiments, compositions described herein are administered topically (including vaginally (e.g., paravaginally), or rectally (e.g., pararectally).

In some embodiments, the present invention features methods and compositions that reduce testosterone (T) levels for treating, ameliorating, and/or preventing progression of dilated cardiomyopathy and heart failure. For example, the reduction in T may preserve systolic function, slow cardiac remodeling, fibrosis and edema, and development of symptomatic HF with reduced ejection fraction (HFrEF).

In some embodiments, the methods and compositions lower circulating T levels. In some embodiments, the methods and compositions may affect biological pathways responsible for cGMP production.

A non-limiting example of a composition that may be used in the methods of the present invention includes gonadotropin-releasing hormone antagonists (e.g., degarelix). The present invention is not limited to GnRH antagonists and includes other medicine classes of androgen (T) suppressants, androgen agonists and androgen receptor-antagonists and androgen synthesis inhibitors and combined androgen blockade by several classes of androgen (T) suppressants.

In some embodiments, the drug is degarelix, abarelix, cetrorelix, ganirelix, elagolix, or relugolix. In some embodiments, the drug is leuprorelin, goserelin, triptorelin, histrelin, or buserelin. The present invention is not limited to the aforementioned drugs or drug classes.

In some embodiments, the present invention comprises a composition for use in treating and/or preventing progression of DCM and HF, the composition comprising a T-inhibiting drug. In some embodiments, the present invention comprises a composition for use in treating and/or preventing progression of dilated cardiomyopathy and heart failure, the composition comprising an androgen-deprivation drug that reduces circulating T levels.

In some embodiments, the present invention comprises a composition for use in improving cardiac contractile function, the composition comprising an androgen-deprivation drug that reduces circulating T levels. In some embodiments, the present invention comprises a composition for use in improving cardiac ejection fraction, the composition comprising an androgen-deprivation drug that reduces circulating T levels.

In some embodiments, the present invention comprises a composition for use in reducing pulmonary and/or systemic edema, the composition comprising an androgen-deprivation drug that reduces circulating T levels. In some embodiments, the present invention comprises a composition for use in reducing cardiogenic edema, the composition comprising an androgen-deprivation drug that reduces circulating T levels. In some embodiments, the present invention comprises a composition for use in reducing plasma norepinephrine levels, the composition comprising an androgen-deprivation drug that reduces circulating T levels. In some embodiments, the present invention comprises a composition for use in reducing plasma renin activity levels, the composition comprising an androgen-deprivation drug that reduces circulating T levels. In some embodiments, the present invention comprises a composition for use in reducing pro-renin receptor levels, the composition comprising an androgen-deprivation drug that reduces circulating T levels.

In some embodiments, the present invention comprises a composition for use in increasing plasma atrial natriuretic peptide (ANP) levels and plasma N-terminal ANP peptide levels, the composition comprising an androgen-deprivation drug that reduces circulating T levels. In some embodiments, the present invention comprises a composition for use in increasing at least one of plasma brain (b-type or BNP) natriuretic peptide levels and plasma N-terminal pro BNP levels, the composition comprising an androgen-deprivation drug that reduces circulating T levels. In some embodiments, the present invention comprises a composition for use in increasing cGMP levels. In some embodiments, increasing cGMP levels using the compositions of the present invention reduces HF symptoms.

In some embodiments, the drug is selected from at least one of a gonadotropin releasing hormone (GnRH) agonist, a GnRH antagonist, an androgen agonist, an androgen antagonist, an androgen-receptor antagonist, and an androgen synthesis inhibitor. In some embodiments, the drug is abarelix, cetrorelix, degarelix, ganirelix, elagolix, or relugolix. In some embodiments, the drug is leuprorelin, goserelin, triptorelin, histrelin, or buserelin. In some embodiments, the drug comprises multiple drugs from the aforementioned drugs and/or classes of drugs.

In some embodiments, the present invention comprises a method for treating and/or preventing progression of dilated cardiomyopathy and heart failure in a subject in need thereof, said method comprising: administering to the subject a therapeutically effective amount of a drug comprising gonadotropin releasing hormone (GnRH) agonist, a GnRH antagonist, an androgen agonist, an androgen antagonist, or an androgen-receptor antagonist, wherein the GnRH agonist, GnRH antagonist, androgen agonist, androgen antagonist, or androgen-receptor antagonist reduces circulating T levels and treats and/or prevents DCM progression and HF in the subject.

In some embodiments, the androgen antagonist is abarelix, cetrorelix, degarelix, ganirelix, elagolix, or relugolix. In some embodiments, the GnRH agonist is leuprorelin, goserelin, triptorelin, histrelin, or buserelin.

In some embodiments, the present invention comprises a method of treating and/or preventing progression of DCM and HF, the method comprising administering a therapeutically effective amount of a T-inhibiting drug to a subject in need thereof.

In some embodiments, the present invention comprises a method of treating and/or preventing progression of DCM and HF, the method comprising administering a therapeutically effective amount of an androgen-deprivation drug that reduces circulating T levels to a subject in need thereof.

In some embodiments, the present invention comprises a method of improving cardiac contractile function, the method comprising administering a therapeutically effective amount of an androgen-deprivation drug that reduces circulating T levels to a subject in need thereof.

In some embodiments, the present invention comprises a method of improving cardiac ejection fraction, the method comprising administering a therapeutically effective amount of an androgen-deprivation drug that reduces circulating T levels to a subject in need thereof.

In some embodiments, the present invention comprises a method of reducing pulmonary and/or systemic edema, the method comprising administering a therapeutically effective amount of an androgen-deprivation drug that reduces circulating T levels to a subject in need thereof.

In some embodiments, the present invention comprises a method of reducing cardiogenic edema, the method comprising administering a therapeutically effective amount of an androgen-deprivation drug that reduces circulating T levels to a subject in need thereof.

In some embodiments, the present invention comprises a method of reducing plasma norepinephrine levels, the method comprising administering a therapeutically effective amount of an androgen-deprivation drug that reduces circulating T levels to a subject in need thereof.

In some embodiments, the present invention comprises a method of reducing plasma renin activity levels, the method comprising administering a therapeutically effective amount of an androgen-deprivation drug that reduces circulating T levels to a subject in need thereof.

In some embodiments, the present invention comprises a method of reducing pro-renin receptor levels, the method comprising administering a therapeutically effective amount of an androgen-deprivation drug that reduces circulating T levels to a subject in need thereof.

In some embodiments, the present invention comprises a method of increasing plasma atrial natriuretic peptide (ANP) levels and plasma N-terminal ANP peptide levels, the method comprising administering a therapeutically effective amount of an androgen-deprivation drug that reduces circulating T levels to a subject in need thereof.

In some embodiments, the present invention comprises a method of increasing plasma brain (b-type or BNP) natriuretic peptide levels and/or plasma N-terminal pro BNP levels, the method comprising administering a therapeutically effective amount of an androgen-deprivation drug that reduces circulating T levels to a subject in need thereof.

In some embodiments, the drug is selected from at least one of a gonadotropin releasing hormone (GnRH) agonist, a GnRH antagonist, an androgen agonist, an androgen antagonist, or an androgen-receptor antagonist. In some embodiments, the drug is abarelix, cetrorelix, degarelix, ganirelix, elagolix, or relugolix. In some embodiments, the drug is leuprorelin, goserelin, triptorelin, histrelin, or buserelin.

### EXAMPLES

The following are non-limiting examples of the present invention. It is to be understood that said examples are not intended to limit the present invention in any way. Equivalents or substitutes are within the scope of the present invention.

### Example 1

Dilated cardiomyopathy (DCM) is characterized by progressive heart enlargement with a reduced ejection fraction (rEF) and is the primary cause of HFrEF. DCM progresses from ‘at risk’ status (Stage A HF) to rEF and cardiac dilation (pre-HF or Stage B). The decline in systolic function causes cardiac fibrosis, severe clinical symptoms from edema in lungs and peripheral tissues (Stage C HF), breathlessness, fatigue, cachexia, disability (advanced HF, Stage D), and premature death.

Despite advancements in pharmacotherapy associated with survival benefits, HFrEF remains an irreversible condition that may require heart transplantation or mechanical circulatory support.

The present invention provides a novel strategy to prevent progression of DCM and symptomatic HFrEF in subjects with DCM by lowering circulating testosterone (T) levels with T deprivation medication(s). Thus, the present invention provides methods and compositions for clinical management of DCM-HFrEF. Without wishing to limit the present invention to any theory or mechanism, it is believed that the benefits of T deprivation are driven in part by the cGMP-regulated pathways.

cGMP is generated intracellularly and diffuses into circulation. cGMP is produced by activation of soluble guanylyl cyclase (sGC) by nitric oxide (NO) and by activation of guanylyl cyclase A (GS-A) by biologically active atrial and B-type natriuretic peptides (ANP/BNP or NPs). Cardiac corin and other enzymes produce active ANP/BNP by cleaving their pro-forms. NPs are degraded by neprilysin (NEP) and cleared by NP receptor C; and cGMP is degraded by phosphodiesterases (PDEs). In DCM, cGMP opposes cardiac dysfunction, remodeling, and fibrosis, and prevents edema and HF symptoms. ANP/BNP, corin, NEP and cGMP are therapeutic targets, and plasma levels of their immunoreactive forms are established biomarkers of HFrEF.

In DCM, classical renin-angiotensin-aldosterone system (RAAS) activation (renin/angiotensinogen-ACE/Ang II/aldosterone axis) compensates for impaired cardiac function. However, its chronic activation has deleterious effects on cardiac structure and performance, leading to edema and HFrEF symptoms. In chronic HF, a decreased ratio of plasma cGMP/NP levels indicates attenuation of the compensatory activity of the cardiac NP system.

Progression to symptomatic HFrEF in DCM is related to cGMP deficiency, which contributes to systolic dysfunction, remodeling, fibrosis, and dysregulation of sodium-water homeostasis leading to edema and HF symptoms. Plasma T and cGMP levels are inversely related, and NPs-cGMP pathway impairment is linked to increased androgenicity. Plasma T stimulates angiotensinogen expression and PRA, which triggers Angli generation, thus blocking cGMP production.

Inventors have found that T deprivation delays the progression of cardiac dysfunction and HFrEF development defined by edema, and prolonged survival in male mice with progressive DCM-HFrEF.

The present invention features methods for preventing or slowing down HFrEF progression in DCM by lowering circulating T levels.

The present invention features methods for utilizing T deprivation initiated at HF A-B stages to support a new shift toward early secondary prevention, rather than starting medical management after symptomatic HF (Stages C-D).

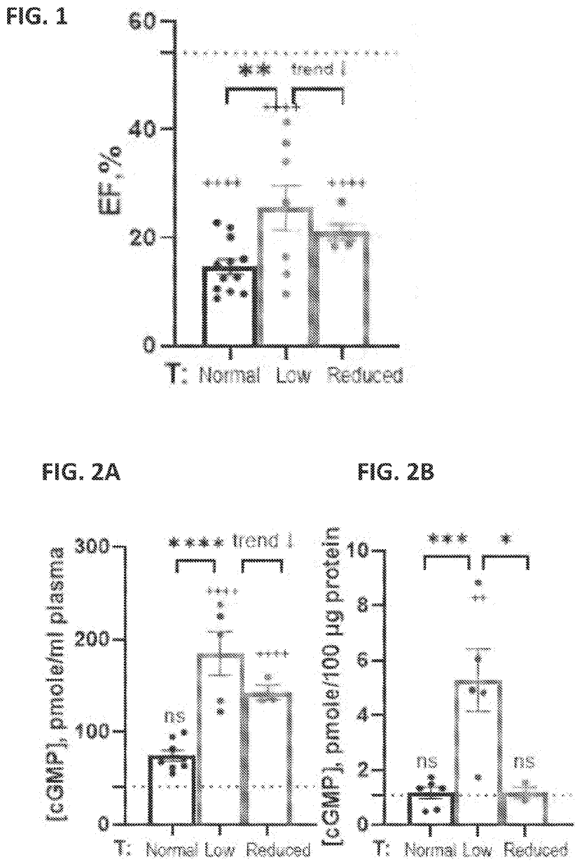

Referring to FIG. 1, T suppression in male DCM mice (low level) by surgical castration (initiated at HF stage A) improves systolic dysfunction vs. non-castrated DCM group (normal level). Partial restoration of plasma T levels (reduced level) in castrated DCM mice (implanted pellets for 3 months, as mice progressed from “at-risk” to advanced HF) showed a trend toward reduced EF.

Surgical T deprivation (castration) is a common technique to investigate the effect of T deficiency in animal models. However, this approach is invasive, irreversible, might be associated with behavioral alterations and does not translate to clinical practice. Androgen deprivation therapy significantly lowers T circulating levels or biological activity and is the standard reversible medical pharmacological approach in patients with advanced prostate cancer, or for adolescents presenting with precocious puberty.

The present invention aims to utilize existing androgen (T)-deprivation drugs for treatment and/or prevention of DCM progression and HF. To suppress T, T deprivation drugs such as but not limited to degarelix (gonadotropin-releasing hormone antagonist) may be used, which reversibly suppresses T production and lowers circulating levels (medical castration) to surgical castration level without T surge and clinical side effects.

The present invention provides preclinical proof-of-concept for the proposed T deprivation strategy as a novel treatment to prevent DCM progression and symptomatic HFrEF in DCM. Referring to FIG. 1, partial restoration of T in the castrated DCM mice caused the reduction of EF suggests that medical T deprivation is beneficial. Thus, degarelix treatment slows the progression of systolic dysfunction (FIG. 4A) and attenuates cardiac remodeling (FIG. 4B), pulmonary edema (FIG. 4C) and pleural effusion (FIG. 4D) in DCM male mice and potentially female mice when analyzed at mouse age corresponding to the previously identified Stage D HF in the untreated DCM groups. Degarelix treatment may also attenuate cardiac fibrosis and cachexia.

Inventors demonstrated that in DCM, circulating T is associated with decreased cGMP plasma and cardiac levels and progressive development of HFrEF. Inventors have found that in DCM-HFrEF, plasma T negatively modulates cardiac contractility, promoting remodeling and edema, but underlying molecular mechanisms of protecting T deprivation remain unclear. Preliminary data demonstrate that in male DCM mice, significant EF raise by surgical castration (Low T: FIG. 1) is associated with significantly increased cGMP levels in plasma and cardiac LV tissue (Low T: FIG. 2A, FIG. 2B). T given back to castrated DCM mice (Reduced T) lowered cGMP levels toward the levels in non-castrated DCM mice (Low T vs. Reduced T: FIG. 2A, FIG. 2B).

### Example 2

The current management of DCM mainly aims at reducing symptoms of already developed HF and improving cardiac function rather than preventing or slowing down DCM progression to symptomatic HF. Treating breathlessness in patients with symptomatic HF is imprecise, costly, and associated with poor outcomes. Novel therapies targeting DCM progression early before the onset of HF are desperately needed. Early treatment of DCM in patients may prevent disease progression to symptomatic HF, reduce the hospitalization rate, and improve outcomes and the quality and duration of the patient's life.

Inventors surprisingly discovered that deprivation of testosterone (T) in male mice with dilated cardiomyopathy (DCM) improves systolic LV function, attenuates heart failure (HF) and prolongs survival in male DCM mice. Inventors further unexpectedly found that T-lowering therapy successfully attenuates DCM progression at the preclinical stages and prevents its progression to symptomatic HF.

Inventors found that plasma T contributes to HF progression in male mice with DCM. In male DCM mice, reduction of plasma T levels by lifestyle modification or surgical castration (initiated at 4 weeks of age corresponding to Stage A HF) improved systolic dysfunction (increased EF and cardiac output), reduced pleural effusion, slowed HF progression to Stage D, and prolonged life.

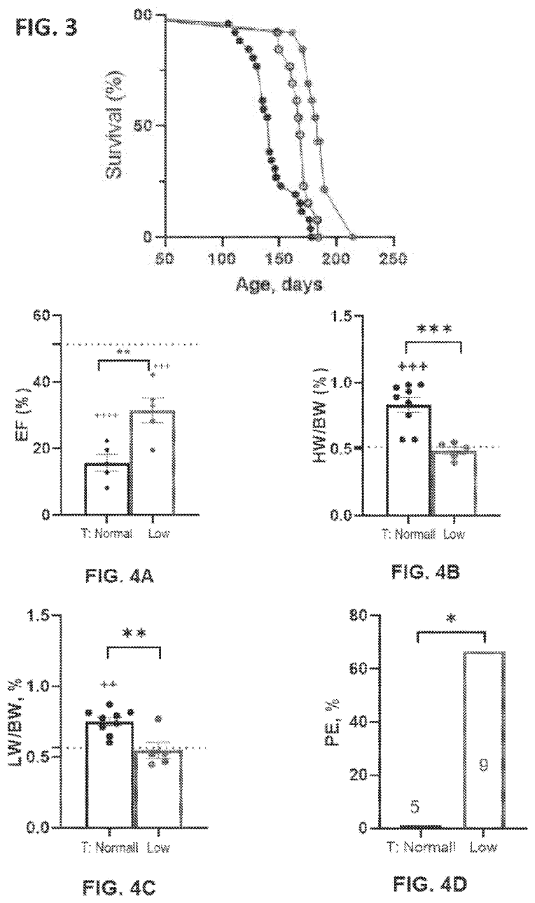

Inventors' data also unexpectedly demonstrates that T lowering by surgical castration of DCM male mice performed at 4 weeks of age, or when mice are at risk for HF (Stage A), or at 7 weeks of age (pre-HF, Stage B) significantly prolonged survival when compared with intact DCM mice (FIG. 3).

Without wishing to limit the present invention to any particular theory or mechanism, T can affect cardiac contractility by acting directly on cardiac cells through the androgen or the estrogen receptors (after aromatization into estradiol), by modulating intracellular calcium-ion homeostasis and apoptosis, or through yet unknown mechanisms. Inventors found that T reduction by surgical castration did not alter androgen and estrogen receptor transcripts levels in LV of DCM mice (4-5 mice/group). T reduction also did not modulate levels of transcript related to calcium homeostasis and apoptosis pathways, but significantly reduced levels of myosin heavy chain transcript Myh7 and its upstream transcription coactivator myocardin, which in clinical and translational HF are related to improved LV function.

Thus, repurposing of T deprivation (lowering) pharmacological therapy for treatment of DCM and HF and/or prevention of HF development in DCM is a novel contribution of the present invention. T deprivation (lowering) pharmacological therapy significantly lowers T plasma levels or biological activity, and is the standard reversible medical pharmacological approach in patients with advanced prostate cancer, for adolescents presenting with precocious puberty, and in gender-affirming hormone therapy.

The results of inventors' studies thus provide preclinical proof-of-concept for the T deprivation strategy as a novel treatment to treat and/or prevent symptomatic HF in DCM. For T deprivation (lowering) therapy, inventors used degarelix (Firmagon@, gonadotropin-releasing hormone antagonist), which reversibly suppresses T production and lowers circulating levels (medical castration) to surgical castration level without T surge and clinical side effects in humans and mice. Inventors chose this androgen antagonist drug vs. androgen agonists and androgen-receptor antagonists as its action in humans and mice grossly mimics surgical castration and does not cause elevation of circulating T levels in response to treatment (feedback loops) and does not affect muscle mass. Inventors also considered that LV transcripts of T receptors were not altered by surgical castration of DCM mice.

To gain translational insights relevant to human DCM-HF, inventors analyzed the outcome of T-lowering therapy in a well-established mouse progressive DCM model on C57BL/6J strain with defined age-related HF stages (time points) for evaluation of systolic function, edema, blood sample collections, etc., in studies of male and female DCM mice. This animal model is normotensive and without kidney dysfunction, and arrhythmia, which excludes variables of blood pressure, arrhythmias, and kidney dysfunction.

Inventors' data surprisingly demonstrates the efficiency of T-lowering (deprivation) pharmacological therapy in slowing DCM progression and development of symptomatic HF in male preclinical DCM-HF model in vivo studies. Inventors developed a dosing strategy in which treatment with degarelix significantly improved cardiac function in the treated group vs. the control untreated group. Treatment was initiated at 4 weeks of age when mice were at risk for DCM. The EF was analyzed at 20 weeks when DCM untreated mice progressed to Stage D HF. The treatment significantly improved EF in DCM mice when compared to the untreated DCM group (FIG. 4A). This treatment also elevated EF in males WT vs. untreated WT group (Mann-Whitney test: P<0.01).

Considering the role of T in muscle growth, T-lowering treatment expectedly reduced pathologically elevated heart-to-body weight ratio (P<0.001, FIG. 4B).

Unexpectedly, treatment with T-lowering drug degarelix reduced pulmonary edema as demonstrated by reduction of the body weight ratios (pulmonary edema, P<0.01, FIG. 4C) and pleural effusion (P<0.05, FIG. 4D).

Importantly, treatment with degarelix did not influence body weights and motor function in male and female mice with DCM and WT littermates.

The effects of T-lowering in DCM on HF-related plasma biomarkers were also explored by the inventors. In DCM, chronic activation of the sympathetic nervous system (SNS) and renin-angiotensin-aldosterone system (RAAS) and dysregulation of the natriuretic peptide system promotes cardiac dysfunction. It leads to the development of symptomatic HF defined by pathological salt-water retention (edema) in the lungs and other tissues. Targeting RAAS is implicated in established DCM-HF pharmacological treatments. Inventors have found that pharmacological normalization of plasma renin activity (PRA) and plasma pro-renin receptor levels ((P)RR) improves systolic function and attenuates HF in DCM male mice.

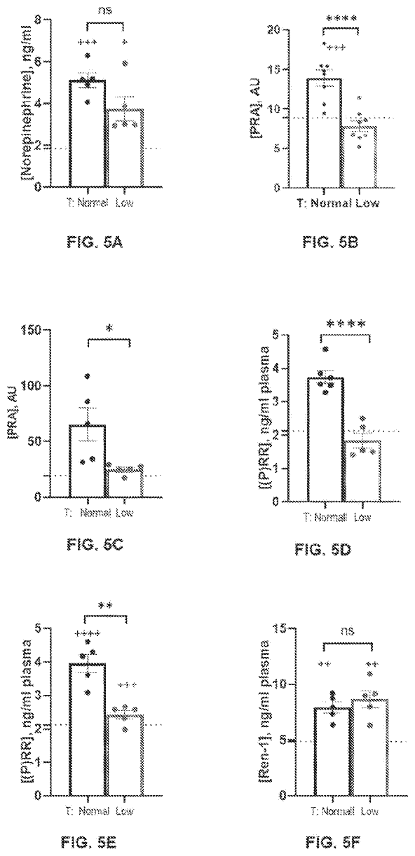

Inventors' data demonstrates that surgical and pharmacological T deprivation significantly reduces PRA and (P)RR levels (markers of RAAS, FIG. 5 B-E). Pharmacological T deprivation also considerably reduced plasma levels of norepinephrine (SNS marker, FIG. 5A). At the same time, pharmacological T deprivation did not affect total renin levels in circulation (FIG. 5F).

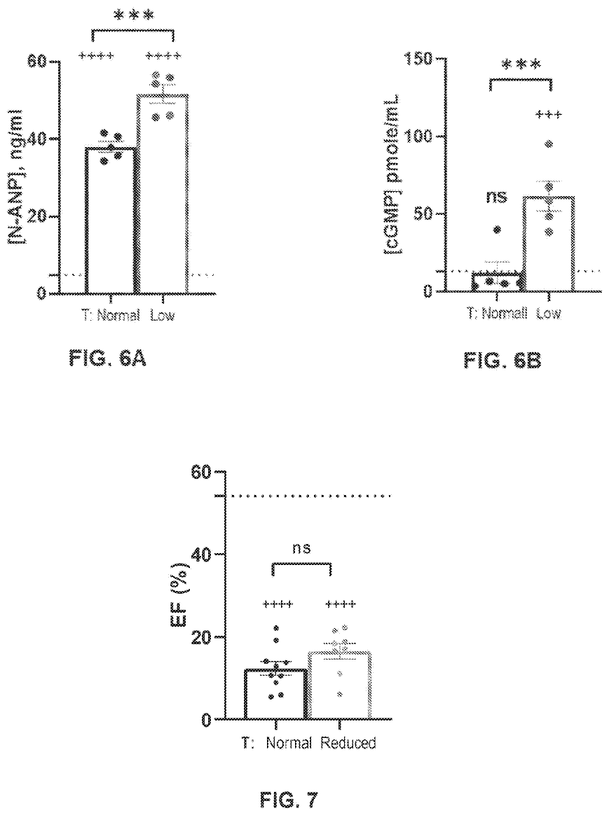

Strikingly, while improving DCM and HF, pharmacological T deprivation significantly increased plasma levels of ANP (measured as NT-ANP) (FIG. 6A) and cGMP (FIG. 6B). This treatment also elevated ANP and cGMP plasma levels in the male's WT vs. untreated WT group (P<0.001, Mann-Whitney test).

Without wishing to limit the present invention to any particular theory or mechanism, these results suggest that in DCM, T might directly or indirectly enable the initiation of cardiomyopathy in part by increasing levels of norepinephrine and or plasma renin activity. T-lowering treatment suppresses norepinephrine and plasma renin activity and will prevent the early development of cardiomyopathy and HF in patients who are at risk.

Further, T-lowering therapy may benefit female DCM subjects like males. Women most of their lives have higher physiological circulating T levels than estrogen. As high T levels in women are associated with adverse cardiovascular events, T-lowering therapy may benefit female DCM subjects like males.

In women, T is produced in the ovary, adrenal gland, and peripheral tissues from the various precursors produced in the ovaries and adrenal gland. Inventors have found that surgical removal of ovaries (ovariectomy, initiated at 4 weeks of age corresponding to Stage A HF) reduces plasma estrogen levels and simultaneously partially reduces plasma T levels, but does not significantly modulate the heart-weight to body-weight ratio, LV dysfunction assessed by EF(%), lung edema, and survival when compared to non-ovariectomized DCM mouse group at 13 weeks of age (Stage D HF in DCM intact mice).

Considering ovarian production of T, inventors evaluated how ovariectomy modulated LV contractility in female DCM mice. Partial reduction of plasma T levels in female DCM mice (low level, ˜55%) by ovariectomy (initiated at HF Stage A) had no significant effect on LV systolic function (although trended towards being increased) vs. DCM intact females (FIG. 7) at 13 weeks of age (Stage D HF). However, this neutral outcome may represent the synergetic effect of suppressed estrogen and partially reduced T levels. Inventors expect T-lowering pharmacological therapy to improve LV systolic function in DCM female mice.

As used herein, the term “population” refers to one or more. As used herein, the terms “a,” “an,” “the,” and “said” include both singular and plural uses, and thus include one or more items. For example, the phrases “a microprocessor,” “the microprocessor,” and “said microprocessor” all encompass one or more microprocessors.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. (canceled)

2. A composition for use in treating and/or preventing progression of dilated cardiomyopathy and heart failure, the composition comprising an androgen-deprivation drug that reduces at least one of circulating testosterone levels and circulating testosterone activity.

3. The composition of claim 2, wherein the composition is further effective for use in improving cardiac contractile function and/or cardiac ejection fraction.

4. (canceled)

5. The composition of claim 2, wherein the composition is further effective for use in reducing at least one of cardiogenic edema, pulmonary edema, and systemic edema.

6. (canceled)

7. The composition of claim 2, wherein the composition is further effective for use in reducing plasma norepinephrine levels, plasma renin activity levels, and/or pro-renin receptor levels.

8. (canceled)

9. (canceled)

10. The composition of claim 2, wherein the composition is further effective for use in increasing at least one of atrial natriuretic peptide levels, N-terminal atrial natriuretic peptide levels, plasma brain (b-type) natriuretic peptide levels, and plasma N-terminal pro b-type natriuretic peptide levels.

11. (canceled)

12. The composition of claim 2, wherein the composition is further effective for use in increasing plasma cGMP levels.

13. The composition of claim 2, wherein the drug is selected from at least one of a gonadotropin releasing hormone (GnRH) agonist, a GnRH antagonist, an androgen agonist, an androgen antagonist, an androgen-receptor antagonist, and an androgen synthesis inhibitor.

14. The composition of claim 2, wherein the drug is abarelix, cetrorelix, degarelix, ganirelix, elagolix, or relugolix.

15. The composition of claim 2 wherein the drug is leuprorelin, goserelin, triptorelin, histrelin, or buserelin.

16.-19. (canceled)

20. A method of treating and/or preventing progression of dilated cardiomyopathy and heart failure, the method comprising administering a therapeutically effective amount of an androgen-deprivation drug to a subject in need thereof, wherein the androgen-deprivation drug reduces at least one of circulating testosterone levels and circulating testosterone activity.

21. The method of claim 20, wherein the method further improves cardiac contractile function and/or cardiac ejection fraction.

22. (canceled)

23. The method of claim 20, wherein the method further reduces at least one of cardiogenic edema, pulmonary edema, and systemic edema.

24. (canceled)

25. The method of claim 20, wherein the method further reduces plasma norepinephrine levels, plasma renin activity levels, and/or pro-renin receptor levels.

26. (canceled)

27. (canceled)

28. The method of claim 20, wherein the method further increases at least one of atrial natriuretic peptide plasma levels, N-terminal atrial natriuretic peptide plasma levels, plasma brain (b-type) natriuretic peptide levels, and plasma N-terminal pro b-type natriuretic peptide levels.

29. (canceled)

30. The method of claim 20, wherein the method further increases plasma cGMIP levels.

31. The method of claim 20, wherein the drug is selected from at least one of a gonadotropin releasing hormone (GnRH) agonist, a GnRH antagonist, an androgen agonist, an androgen antagonist, an androgen-receptor antagonist, and an androgen synthesis inhibitor.

32. The method of claim 20, wherein the drug is at least one of abarelix, cetrorelix, degarelix, ganirelix, elagolix, and relugolix.

33. The method of claim 20, wherein the drug is at least one of leuprorelin, goserelin, triptorelin, histrelin, and buserelin.

34. A composition for use in treating and/or preventing progression of dilated cardiomyopathy and heart failure, the composition comprising a testosterone-inhibiting drug, wherein the drug is selected from at least one of a gonadotropin releasing hormone (GnRH) agonist, a GnRH antagonist, an androgen agonist, an androgen antagonist, an androgen-receptor antagonist, and an androgen synthesis inhibitor.

35. A method of treating and/or preventing progression of dilated cardiomyopathy and heart failure, the method comprising administering a therapeutically effective amount of a composition according to claim 34.
